(19) United States
(12) Patent Application Publication
(10) Pub. No.: US 2019/0156928 A1
(43) Pub. Date: May 23, 2019

(54) SYSTEM AND METHOD FOR ASSISTING MEDICATION MANAGEMENT AND ADHERENCE

(71) Applicants: Shuohui Chen, San Francisco, CA (US); Tianyi Tang, San Francisco, CA (US); Remo L. Morelli, San Francisco, CA (US)

(72) Inventors: Shuohui Chen, San Francisco, CA (US); Tianyi Tang, San Francisco, CA (US); Remo L. Morelli, San Francisco, CA (US)

(21) Appl. No.: 15/449,967

(22) Filed: Mar. 5, 2017

Related U.S. Application Data
(60) Provisional application No. 62/444,834, filed on Jan. 11, 2017.

(51) Int. Cl. G16H 20/10; G06F 9/54; G06F 16/27; H04L 29/06; G06N 5/04; G06N 7/00

(52) U.S. Cl. CPC G16H 20/10; G06F 9/547; G06N 7/005; H04L 63/10; G06N 5/04; G06F 16/27

(57) ABSTRACT

An affordable and convenient system that a patient and their medication related parties can use to monitor their adherence to multiple medications in their original pill bottles. The high precision scale sensor and the firmware collects, checks and stores a users' medication information in a backend data platform in the cloud through a secured channel. A mobile app serves as a personal virtual assistant that relies on up-to-date artificial intelligence and messaging technology to set up a medication adherence monitoring system and secured communication platform among the related parties to the patient.

### CROSS REFERENCE TO RELATED APPLICATIONS

This application claims the benefit of priority under 35 USC § 119(e) to U.S. Provisional Patent Application Ser. No. 62/444,834, filed on Jan. 11, 2017 and titled: “SYSTEM AND METHOD FOR ASSISTING MEDICATION MANAGEMENT AND ADHERENCE,” the entire disclosure of which is hereby incorporated by reference in its entirety for all purposes.

### TECHNICAL FIELD OF THE INVENTION

The disclosure generally relates to managing prescription medications, and more particularly to systems and methods for assisting in medication management and adherence of prescription medications.

### BACKGROUND OF THE INVENTION

Patient non-adherence to prescription medication is a major medical problem. It has been estimated that in the U.S. between $100 and $300 billion of avoidable health care costs are attributed to non-adherence of prescription medication, which represents 3% to 10% of the total U.S. health care costs. Patents that overdose on their prescription medication are another medical problem that increases morbidity and mortality rates.

Many systems and methods have been developed to address these problems. However, the current adherence systems have high cost, require additional manual effort to set up and maintain them, cause excessive wastes or tracking errors when a prescription changes, or are non-portable. Further, the current adherence systems do not conveniently support health care providers, such as doctors or pharmacists, or their social interactions among them and their patients based on the updated patients medication adherence situation. Additionally, few current adherence systems support the original packaging for medication using pill bottles. In addition, no current medication adherence systems have taken advantage of the rapidly developing technologies especially artificial intelligence and deep learning to better assist patient adherence and facilitate each user in this system.

Therefore, there remains a need for inexpensive, intelligent and socially connected systems and methods to assist patients in adherence to their prescribed medication

### SUMMARY OF THE INVENTION

To address these needs, the disclosure provides systems and methods for assisting in medication management and adherence of prescription medications through a docking device on which multiple pill bottles can be placed. The docking device includes multiple high resolution scale sensors, a microcontroller board with wireless connectivity modules and firmware program, an alerting module, a battery and a USB connector, and other customized sensors such as a temperature sensor.

The disclosure further provides mobile and web apps that are provided to patients, their families and caregivers. These apps communicate with the cloud through authorized application programming interfaces (APIs) that provide the following functions:

First, the apps provide for the initiation of new docking devices and/or new pill bottles for patients or caregivers. For convenient and safe usage, the app may take users' inputs through texts, photos or voices for initialization. Understanding of users' inputs and automatically initializing the docking devices and/or the pill bottles are performed through self-trained machine learning models or third-party machine learning APIs.

Next, the apps provide for the monitoring when each dose is taken in real-time and when the medication storage temperature is normal, e.g. room temperature.

The apps also provide for reminding the patients and/or caregivers when and how to take pills safely.

The apps further provide alerts to the patients and/or caregivers via beeping and app messages when wrong doses or medications are taken or when the environment temperature is not ideal.

Finally, the apps provide a summary of patient medication adherence and share with authorized users via email, text or charts.

The disclosure also provides mobile and web apps provided to the physicians or pharmacists of the patients to share the patients' authorized medication adherence history and communicate on treatments or prescriptions via real-time chat, text or phone calls, etc.

The disclosure further provides apps for real-time chatting and communicating function among each related parties via authorized text and figures, which facilitate some scenarios, such as when a physician makes changes on the prescription for one of their patients, they may use the app to communicate with the patient or the caregivers on this change and check following adherence.

### BRIEF DESCRIPTION OF THE DRAWINGS

FIG. 1 illustrates an embodiment of the components of a system for assisting medication management and adherence as described herein;

FIG. 2 illustrates an embodiment of an initialization process for a system for assisting medication management and adherence as described herein;

FIG. 3 illustrates an embodiment of how a patients' medication adherence information is shared across the patient and their physicians which enables social network among the related parties as described herein; and

FIG. 4 illustrates an embodiment of a docking device and its components as described herein.

### DETAILED DESCRIPTION OF THE EMBODIMENTS

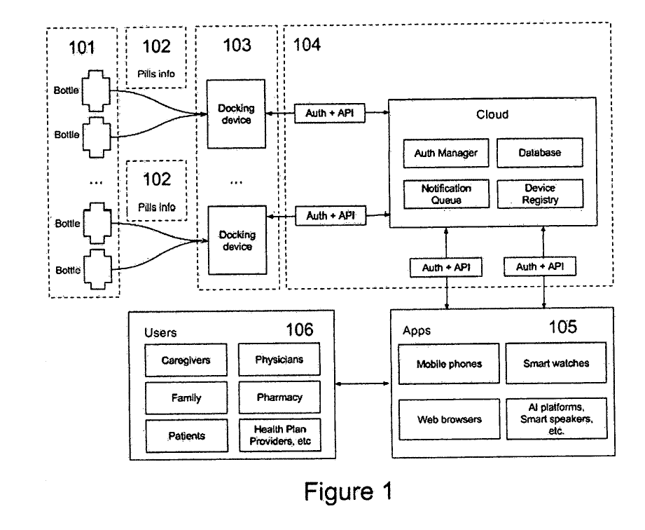

FIG. 1 illustrates an embodiment of a system for assisting medication management and adherence for a user. The various components as shown in this figure include one or more bottles (101), which can be placed onto a docking device (103). In an embodiment, the one or more bottles (101) can be or more pill bottles that contain a prescription and/or non-prescription medication in pill form. The one or more bottles (101) can be kept on the docking device before and after the user takes the medication.

As shown in FIG. 1, the pill related information (102), which can be in terms of bottle mass or weight change and the like, e.g. when a user removes a pill for their dosage regiment, can be read by a microcontroller board (not shown) present in the docking station (103), authenticated and posted to a cloud database (104) through an API. In one embodiment, the API can be a RESTful API with authorization tokens. As shown in this figure, the cloud (104) can include an authorization manager, a database, a notification queue, and a device registry. Similarly, the system can then send a notification relating to the pill related information (102) from the cloud (104) via a text or app message (105) to the user, including but not limited to users such as caregivers, physicians, family, pharmacy, patients, and health care providers (106).

Once the device and bottles are initialized, the user is required to keep the one or more bottles (101) on the docking device (103) before and after they take the medication. Initializing the docking device and each new bottle enables the user to input all the required user and medication information with minimum amount of effort. With the rapid progress of artificial intelligence, GPUs and big data infrastructure, etc., multiple platforms provide machine learning or AI (artificial intelligence) APIs or open-source programs that can perform high quality AI tasks such as image recognition, OCR (Optical Character Recognition), NLP (natural language processing), voice recognition, speech and translation, etc. These techniques are used in this invention to assist users setting up their devices and regular use of the device.

When the third-party APIs are not available, training and maintaining artificial intelligence or machine learning models for these tasks may be conducted through research.

The cloud database includes but is not limited to the following features:

1) Leverage the server-less computing techniques to perform a real-time and relatively scalable service in data retrieval and posting, etc.

2) Leverage industry-standard protocol for authorization and end-to-end encryption to protect the security of users' medication data.

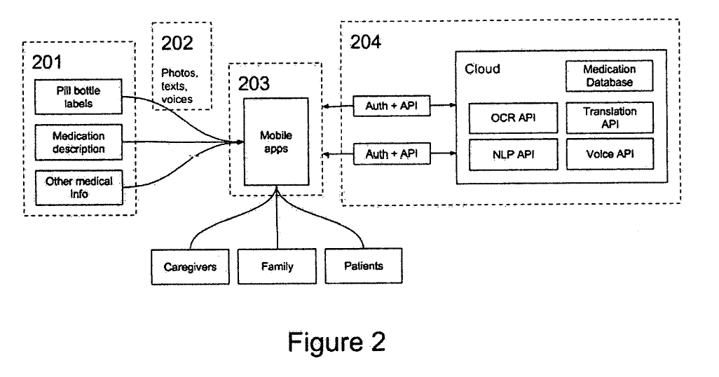

FIG. 2 illustrates an embodiment of an initialization process for the system for assisting medication management and adherence. With the above system initialization goal and the availability of machine learning APIs, the app (203) can communicate with either third-party or self-trained machine learning models via APIs (204), and can function like a virtual assistant to take the users' minimum inputs, such as the NDC package number, Rx, the label of the bottle or the doctor's prescriptions, etc. (201) via texts, photos or voices (202). It parses the input and automatically initializes the system and/or each new pill bottle. Users can also review and manually revise any information if they find the machine generated medication information is incorrect.

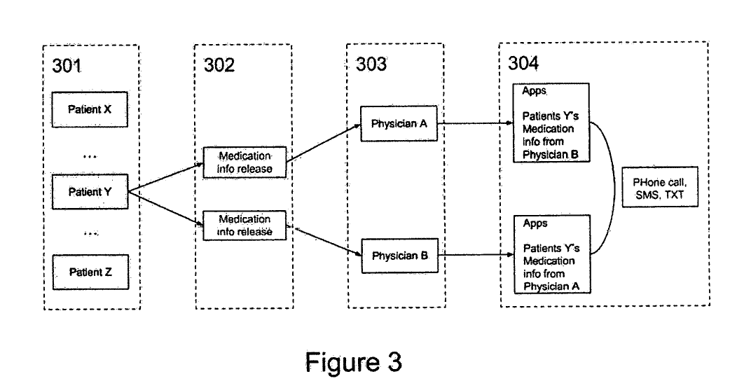

FIG. 3 illustrates an embodiment of how a patients' medication adherence information is shared across the patient and their physicians which enables social network among the related parties. As shown in this figure, the system for assisting medication management and adherence for a user also provides a convenient way to communicate across the network (304) of patients (301), caregivers, families and physicians (303). With minor system configuration, this network can be extended to other medical plan providers such as pharmacists and insurance agents, etc., after they install the app described herein, and receive the patients' release agreement of their medication adherence history (302).

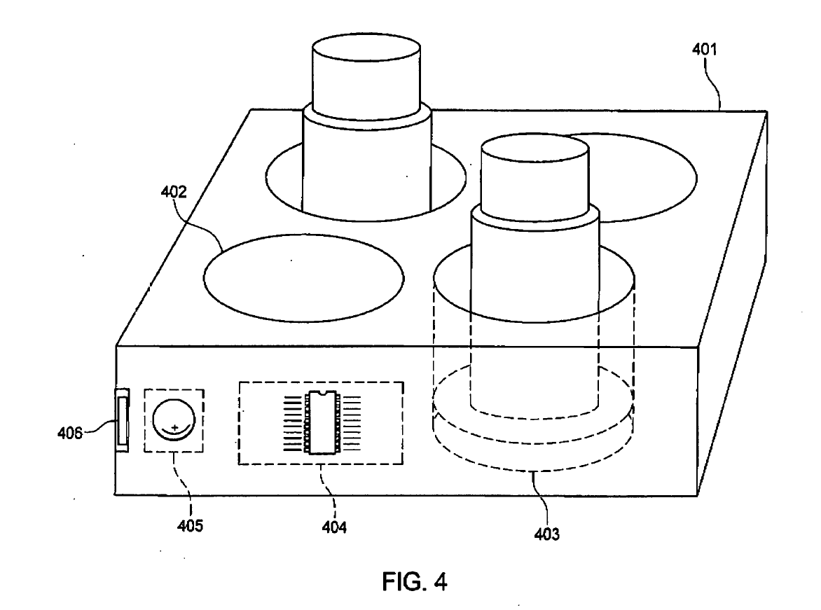

FIG. 4 illustrates an embodiment of a docking device (401) and its components, on which multiple pill bottles can be placed in slots (402). In this figure, the device includes one or more multiple high resolution scale sensors (403), which measures the mass or weight of the pill bottles; and a microcontroller board (404) with wireless connectivity modules configured for Wi-Fi, Cellular, or Bluetooth wireless protocol.

Firmware program is loaded in the memory of the microcontroller board. It includes the following but not limited to the following modules:

1) Measurement: processes the sensors' signals and maintains the accuracy of the scales through auto-retaring and auto-resetting the scales, auto-adjusting the measurement based on feedback control rules and models.

2) Monitoring: checks and monitors the pill changes by measuring the bottle's mass or weight change. Specifically, train and keep updating a Bayesian inference model using data including the regular pills' mass and measurements changes from the scales, etc. The model runs on firmware and its coefficients are stored in local microcontroller board and synced with the cloud database. Then the model can be used to compute the probability of an object moving in or out of a bottle being a pill.

3) Denise: filter out noise on the bottle's measured mass or weight caused by issues such as tilt, shake of the docking device, moving non-pill objects in or out of a bottle, moving bottles to different scales after taking a pill, etc. If the noise cannot be automatically filtered, such as large tilt, generate warning message through beep or apps and allow users to manually re-set the bottles.

4) Security: use industry-standard protocol protect users' privacy, including end-to-end encrypting and decrypting medication messages at both local microcontroller board and cloud database; storing and refreshing tokens or security keys for authentication.

5) Communication: set up secured wireless connections to the Internet or other devices through low-power Internet of Things (IoT) networks, including but not limited to Wi-Fi, Bluetooth, Cellular, etc.

6) Messaging: posts and get messages to or from the cloud database in HTTP Secure (https) protocol or other secure protocols through wireless Internet connection. It also includes controlling a mini speaker and a LED/LCD light installed on the docking device to beep, alarm or display information in some regular or special situations, such as time or overtime for taking medications, overdose detected, ill-positioned device or bottles, etc.

7) Extensions: read signals from other simple sensors connected to the device, such as temperature sensor; support plug-and-play for using additional external scale sensors to perform the above firmware functions.

The docking device (401) also includes a battery (405) and a USB connector (406) for power supply.

A firmware program can be loaded in the memory of the microcontroller board (404). It processes the sensor signals, checks the pills changes, generates authorized pills change messages in designed connectivity protocol, sends the message to the cloud database through wireless connection, pulls necessary authorized sensor and bottle information from cloud database, and receives OTA (over-the-air) packages to upgrade the old version, etc.

As illustrated in FIG. 4, there are two slots in the docking device (401) for the pill bottles, but the device can have from 1 to 5 or 6 slots because some users take 5 or more medications each month. In fact, 1 out of 6 people in the U.S. aged 45-64 years old take at least 5 drugs each month; and this fraction is expected to grow up to 2 out of 5 for U.S. people of at least 65 years old.

The following functions are supported by the system:

First, the apps provide for the initiation of new docking devices and/or new pill bottles for patients or caregivers. For convenient and safe usage, the app may take users' inputs through texts, photos or voices for initialization. Understanding of users' inputs and automatically initializing the docking devices and/or the pill bottles are performed through self-trained machine learning models or third-party machine learning APIs.

The following basic functions are supported by the system and app:

Organize the pills into virtual pill packs by their scheduled or programmed medication time. By default, the pills can be roughly packed into AM, PM, evening and Night four packs. However, for some medications which need to take together and/or are relatively time-sensitive or time-critical for taking, and/or whose pills or doses need to be well controlled and varied by time, the fully controlled virtual pill packs may provide better medication adherence.

Next, the system and apps provide for the monitoring when each dose is taken in real-time and when the medication storage temperature is normal.

The system and apps also provide for reminding the patients and/or caregivers when and how to take pills safely.

The system and apps further provide alerts for the patients and/or caregivers via beeping and app messages when wrong doses or medications are taken or when the environment temperature is not ideal.

Finally, the system and apps provide a summary of patient medication adherence and share with authorized users via email, text or charts.

The docking device and the app are designed to serve as a virtual assistant to users through up-to-date artificial intelligence techniques. The app can be integrated, and launched and available on multiple platforms, including mobile phones, web browsers, wearable devices, smart speakers, as well as other AI powered virtual assistant agents, platforms and devices. Therefore, the user may interact with the device and the app through multiple ways, and further gets connected to their related parties.

Portability is also a feature of the docking device. First the size of the hardware will be small enough for carrying conveniently by travelers; second, the user may use the standard containers or packages, which may be purchased together with the device originally from the manufacturer, to carry their pills; and third, the app can be configured into “travel” mode to allow the users to manually check in their medication status.

The network platform has many benefits to the patient. For example, when they need to visit multiple doctors, each doctor may quickly pull out all their medication and medication adherence history; and when needed and agreed to by the patients, both doctors may leave notes on the patient's medication history through the app and make real-time chat via text and images on this app.

In another situation when a physician makes changes on their prescription to one of their patients, they may use the app to communicate with the patient or the caregivers on this change and then use this app to check the new medication adherence.

It should be readily understood by those persons skilled in the art that the present disclosure is susceptible of a broad utility and application. Many embodiments and adaptations of the present disclosure other than those herein described, as well as many variations, modifications and equivalent arrangements can be apparent from or reasonably suggested by the present disclosure and the foregoing description thereof, without departing from the substance or scope of the present invention. Accordingly, while the present disclosure has been described herein in detail in relation to its embodiments, it is to be understood that this disclosure is only illustrative and exemplary of the present disclosure and is made merely for purposes of providing a full and enabling disclosure of the invention. The foregoing disclosure is not intended or to be construed to limit the present disclosure or otherwise to exclude any such other embodiment, adaptations, variations, modifications and equivalent arrangements, the present disclosure being limited only by the claims appended hereto and the equivalents thereof.

What is claimed is:

1. A system for assisting management and adherence of medication, comprising:
a docking device having:
one or more slots where one or more pill bottles containing one or more pills can be placed;
one or more high resolution scale sensors for measuring a mass of the one or more pill bottles;
one or more microcontroller boards having one or more firmware programs and one or more wireless connectivity modules,
wherein the one or more firmware programs monitors a change in mass of the one or more pill bottles based on the measured mass of the one or more pill bottles, and computes a probability of an object moving in or out of the one or more pill bottles being one or more pills based on a Bayesian inference model,
wherein pill bottle related information and the probability are read by the one or more microcontroller boards and posted to a cloud database through the one or more wireless connectivity modules using a first RESTful application programming interface (API) with authorization tokens; and
one or more mobile or web apps for communicating with the cloud database through a second RESTful API with authorization tokens.

2. The system for assisting management and adherence of medication of claim 1, wherein the one or more pill bottles is one or more prescription medicine pill bottles.

3. The system for assisting management and adherence of medication of claim 1, wherein the one or more wireless connectivity module is configured for a Wi-Fi, cellular, or Bluetooth wireless protocol.

4. The system for assisting management and adherence of medication of claim 1, wherein the one or more firmware programs are loaded into a memory of the one or more microcontroller boards.

5. The system for assisting management and adherence of medication of claim 4, wherein the one or more firmware programs processes sensor signals, checks pill changes, generates authorized pill change messages in a designed connectivity protocol, sends messages to the cloud database through the wireless connection, pulls necessary authorized sensor and bottle information from the cloud database, or receives over-the-air (OTA) packages to upgrade older versions.

6. The system for assisting management and adherence of medication of claim 1, wherein the cloud database includes an authentication manager, a notification queue, and a device registry.

7. The system for assisting management and adherence of medication of claim 1, wherein the cloud database sends a notification relating to the pill related information via a text or app message to a user through the second RESTful application programming interface (API) with authorization tokens.

8. The system for assisting management and adherence of medication of claim 7, wherein the user is a caregiver, physician, family member, a pharmacy, a patient, or a health care provider.

9. The system for assisting management and adherence of medication of claim 1, further comprising the docking device having one or more temperature sensors.

10. The system for assisting management and adherence of medication of claim 1, wherein the one or more mobile or web apps for communicating with the cloud through the second RESTful API with authorized tokens initializes a new docking device or a new pill bottle for a user through a text, photo or voice message.

11. The system for assisting management and adherence of medication of claim 1, wherein the one or more mobile or web apps for communicating with the cloud through the second RESTful API with authorized tokens monitors when each dose is taken in real-time.

12. The system for assisting management and adherence of medication of claim 1, wherein the one or more mobile or web apps for communicating with the cloud through the second RESTful API with authorized tokens reminds a user when to safely take a pill in the one or more pill bottles.

13. The system for assisting management and adherence of medication of claim 1, wherein the one or more mobile or web apps for communicating with the cloud through the second RESTful API with authorized tokens provides alerts to a user via beeping or app messages when a wrong dose or medication is taken.

14. The system for assisting management and adherence of medication of claim 1, wherein the one or more mobile or web apps for communicating with the cloud through the second RESTful API with authorized tokens provides a summary of user medication adherence and shares this with the user via email, text or charts.

15. The system for assisting management and adherence of medication of claim 1, wherein the one or more mobile or web apps for communicating with the cloud through the second RESTful API with authorized tokens allows physicians or pharmacists, of the user to share the users' authorized medication adherence history and communicate on treatments or prescriptions via real-time chat, text or phone calls.

16. The system for assisting management and adherence of medication of claim 1, wherein the one or more mobile or web apps for communicating with the cloud through the second RESTful API with authorized tokens allows real-time chatting and communicating among each user via authorized text.

17. A method for assisting management and adherence of medication, comprising:
initiating a docking device and one or more pill bottles for user and medication information through one or more apps, wherein the docking device includes:
one or more slots where one or more pill bottles containing one or more pills can be placed;
one or more high resolution scale sensors for measuring a mass of the one or more pill bottles;
one or more microcontroller boards having one or more firmware programs and one or more wireless connectivity modules,
wherein the one or more firmware programs monitors a change in mass of the one or more pill bottles based on the measured mass of the one or more pill bottles, and computes a probability of an object moving in or out of the one or more pill bottles being one or more pills based on a Bayesian inference model;
reading pill bottle related information and the probability by the one or more microcontroller boards; and
posting the pill bottle related information and the probability to a cloud database through the one or more wireless connectivity modules using a first RESTful application programming interface (API) with authorization tokens.

18. The method for assisting management and adherence of medication of claim 17, further comprising:
communicating with the cloud database with one or more user mobile or web apps through a second RESTful API with authorization tokens, wherein the user is a caregiver, physician, family member, a pharmacy, a patient, or a health care provider.

19. The method for assisting management and adherence of medication of claim 17, wherein the cloud database sends a notification relating to the pill bottle related information via a text or app message to a user through the second RESTful API with authorization tokens.

20. The method for assisting management and adherence of medication of claim 19, wherein the user is a caregiver, physician, family member, a pharmacy, a patient, or a health care provider.
